(19) United States
(12) Patent Application Publication
(10) Pub. No.: US 2010/0249703 A1
(43) Pub. Date: Sep. 30, 2010

(54) STERILIZABLE VACUUM HANDPIECE

(76) Inventor: John A. Cliff, Oxnard, CA (US)

Correspondence Address: GALLAGHER & DAWSEY CO., L.P.A., P.O. BOX 785, COLUMBUS, OH, 43216, US

(21) Appl. No.: 12/412,445

(22) Filed: Mar. 27, 2009

(51) Int. Cl. A61M 39/22

(52) U.S. Cl. 604/119

(57) ABSTRACT

A sterilizable vacuum handpiece that may include a base unit reversibly coupled to a handle unit, having a central vacuum passage connecting proximal and distal ends. A hand-removable base unit valve may divide a base unit vacuum passage and a hand-removable handle unit valve may separate a handle unit vacuum passage, in adjustable fluid communication with each other. The valves may be retained within bores and counter-bores, and may achieve fluid-tight fit with O-rings retained within the valves. The valves may have vacuum passages with surface areas greater than or equal to a least diameter of the vacuum passage containing the valve. In at least one embodiment, the handle valve is releasably retained by the opposing ends of a “bucket-handle” shaped controller. Alternatively, the handpiece may be formed as a single combined unit, where the terms base unit and handle unit refer only to regions of an otherwise unified structure.

### STATEMENT REGARDING FEDERALLY SPONSORED RESEARCH OR DEVELOPMENT

This invention was not made as part of a federally sponsored research or development project.

### TECHNICAL FIELD

The instant invention relates to a sterilizable vacuum handpiece, particularly to a device for applying suction in medical, dental, and veterinary applications that may be disassembled for cleaning and sterilization.

### BACKGROUND OF THE INVENTION

Surgical suction devices are commonly used in wide variety of surgical procedures in the medical, dental, and veterinary fields. During a surgical procedure, varying amounts of blood, mucus and small surgical debris are produced. The surgical operator needs a means to evacuate these materials, lest they obscure the field of surgery and complicate the procedure. In the dental field, such suction devices are often colloquially known as “handpieces.”

At its simplest, some of the simplest prior art devices are nothing more than a flexible or rigid tube attached to a source of negative pressure, i.e., suction, used manually to evacuate the surgical field. As such, these devices have numerous limitations. If allowed to be in continuous connection to suction, such devices are noisy and distracting, and their continuous suction causes them to adhere themselves to human or animal tissue, possibly causing damage to delicate structures.

At the next level of complexity, surgical suction devices may be supplied with any of a wide variety of side channels in their internal suction channels, so that when such a side channel is left unoccluded, there is little or no suction effect produced at the tip of the device. The operator typically occludes the opening with a thumb or finger, and suction is restored at the device tip. Such devices are also noisy when running with a side-channel open, and require a continuous application of the thumb or finger to occlude the side port in order to produce usable suction in the surgical field, thus potentially robbing the operator of the substantial use of one hand.

These simple devices also pose considerable challenges in the regulation of high and low suction levels. A simple suction tube reflects only the suction level provided at the suction source, so that in a typical aseptic procedure, the surgeon must have another person manipulate the negative pressure regulator of an external suction source. With the side-channel suction device, suction can be somewhat varied by partially to completely occluding the side-channel, but this is inaccurate and manually difficult. A simple slip of the thumb or the finger of a fraction of a millimeter can result in full suction being applied, often at a very inopportune moment.

Valves may be incorporated into the suction device to address the above problems, but these bring their own attendant issues. Valves typically introduce significant narrowing of the diameter of any channel, and in a surgical suction device, such narrowing is an open invitation to clogging. Additionally, the inherent complexity of many valves make such devices difficult to clean and sterilize after use, or conversely, expensive if they are to be disposed of after use.

The various embodiments of the instant invention discussed below, as well as other embodiments as would be known or envisioned by one skilled in the art, address these and other shortcomings of the prior art. Such shortcomings are not elaborated as limitations of any kind, but only as illustrations of some of the possible advantages of one or more embodiments of the instant invention.

### SUMMARY OF THE INVENTION

In its most general configuration, the claimed sterilizable vacuum handpiece advances the state of the art with a variety of new capabilities and overcomes many of the shortcomings of prior devices in new and novel ways. In its most general sense, the handpiece overcomes the shortcomings and limitations of the prior art in any of a number of generally effective configurations. The handpiece demonstrates such capabilities and overcomes many of the shortcomings of prior methods in new and novel ways.

Numerous variations, modifications, alternatives, and alterations of the various preferred embodiments, processes, and methods may be used alone or in combination with one another as will become more readily apparent to those with skill in the art with reference to the following detailed description of the preferred embodiments and the accompanying figures and drawings.

A sterilizable vacuum handpiece generally may include a base unit reversibly coupled to a handle unit, although an embodiment is envisioned wherein the description of “units” merely identifies general regions of an integral, one-piece handpiece body. In one embodiment, the base unit may have a central vacuum passage connecting a base unit proximal end and a base unit distal end. Similarly, the handle unit may have a central vacuum passage connecting a handle unit proximal end and a handle unit distal end.

In at least one embodiment, a hand-removable base unit valve may separate the base unit vacuum passage into a base unit vacuum passage proximal portion in adjustable fluid communication with a base unit vacuum passage distal portion. By hand-removable, it is meant that the normal finger strength of a human operator is sufficient to remove and replace the base unit valve in its position in the base unit. The base unit valve controls adjustable fluid communication between the base unit vacuum passage proximal portion and the base unit vacuum passage distal portion.

Again, in at least one embodiment, a hand-removable handle unit valve may separate the handle unit vacuum passage into a handle unit vacuum passage proximal portion in adjustable fluid communication with a handle unit vacuum passage distal portion. As was the case with the base unit valve, by hand-removable, it is meant that the normal finger strength of a human operator is sufficient to remove and replace the handle unit valve in its position in the handle unit. The handle unit valve controls fluid communication between the handle unit vacuum passage proximal portion and the handle unit vacuum passage distal portion.

One skilled in the art will realize that numerous valve configurations are possible. In one embodiment, the base unit valve may further include a base unit valve bore having at least one base unit valve bore surface extending from the base unit external surface to the base unit vacuum passage. This construction thereby places the base unit external surface in communication with the base unit vacuum passage, with the base unit valve bore reversibly receiving a base unit valve stem having at least one base unit valve stem surface. In such an embodiment, the base unit valve stem is rotatably received within the base unit valve bore, and the base unit valve stem includes an internally formed base unit valve vacuum passage. Rotation of the base unit valve stem may place the base unit vacuum passage proximal portion and the base unit vacuum passage distal portion in adjustable fluid communication.

The base unit valve stem may be releasably retained by friction fit within the base unit valve bore so that all valves may be hand-removable, although one skilled in the art may envision embodiments in which tools are required for valve removal.

In order to maximize a fluid-tight fit between all parts of the instant invention, in some embodiments, the base unit valve stem surface may have at least one base unit valve O-ring retainer sized to contain at least one base unit valve O-ring. It is equally possible for the base unit valve bore surface to have at least one base unit valve O-ring retainer sized to contain at least one base unit valve O-ring.

One skilled in the art will realize that there will be numerous surface area relationships possible between the valves and their corresponding vacuum passages in terms of relative surface area, but in at least one embodiment, the base unit valve vacuum passage has a surface area equal to or greater than 80% of a smallest base unit vacuum passage surface area, and in another embodiment, the handle unit valve vacuum passage may have a surface area equal to or greater than 80% of a smallest handle unit vacuum passage surface area. The term “smallest vacuum passage surface area” is used in recognition of the fact that the vacuum passages may not have a uniform diameter nor a uniform surface area.

One skilled in the art will also realize that numerous possible designs are feasible for the handle valve. In one embodiment, the handle unit valve further includes a first handle unit valve bore having at least one first handle bore surface extending from the handle unit external surface to the handle unit vacuum passage. In such an embodiment, the handle unit valve bore reversibly receives a handle unit valve stem having a handle unit valve stem surface. There may be a second handle unit valve bore having at least one first handle bore surface on an opposing surface of the handle unit from the first handle unit valve bore, extending from the handle unit external surface to the handle unit vacuum passage. Such a second handle unit valve bore may also reversibly receive the handle unit valve stem, and the handle unit valve stem may be rotatably received within the first handle unit valve bore and the second handle unit valve bore.

The handle unit valve stem may have an internally formed handle unit valve vacuum passage, whereby rotation of the handle unit valve stem places the handle unit vacuum passage proximal portion and the handle unit vacuum passage distal portion in adjustable fluid communication. Thus the operator has, in a convenient and ergonomic position, means for making very subtle adjustments in flow through the handpiece on a near continuous basis. To improve hand feel and dexterity while using the handpiece, the base unit valve further comprises a base unit valve finger grip, and the handle unit external surface may have at least one ergonomic gripping means selected from the group of gripping means consisting of finger ridges and at least one thumb projection.

In order to maximize a fluid-tight fit between all parts of the instant invention, in some embodiments, the handle unit valve stem surface may have at least one handle unit valve O-ring retainer sized to contain at least handle unit valve O-ring. Equally possible, the handle unit valve bore surface may have at least one handle unit valve O-ring retainer sized to contain at least one handle unit valve O-ring.

In at least one embodiment, the handle valve is releasably retained by the opposing ends of a “bucket-handle” shaped controller, which, particularly if it is made from a resilient material, may be easily removed to allow for removal of the handle unit valve stem for cleaning. In general, ease of cleaning and sterilization is one of the major features of many of the preferred embodiments of the instant invention. In some embodiments, the valves are hand removable, and may be easily slipped from the handpiece. In other embodiments the handpiece may be further disassembled into the base unit and the handle unit.

In at least one embodiment, the base unit may have at least one base unit proximal end O-ring retainer sized to contain at least one base unit proximal end O-ring. However, as a more general teaching, O-rings held in corresponding retainers encourage fluid-tight seals at the valves while still allowing easy disassembly.

Furthermore, while it may be illustrated in this specification that O-rings and corresponding retainers appear on a particular part of the handpiece, there is no reason why these structures cannot appear on the corresponding parts of the handpiece. For example, while the base unit proximal end is depicted as a male fitting bearing at least one base unit proximal end O-ring retained within at least one base unit proximal end O-ring retainer, there is no reason why the handpiece cannot be configured such that the at least one O-ring and retainer could not be placed on an internal surface of the handle unit vacuum passage. Furthermore, while the base unit is depicted as a male fitting at the base unit proximal end and the handle unit as a female fitting at the handle unit distal end, there is no reason why this relationship could not be inverted, providing a female fitting on the base unit at the base unit proximal end and a male fitting on the handle unit at the handle unit distal end.

Additionally, while in a preferred embodiment the base unit and the handle unit are reversibly coupled in a liquid-tight fit, it may be desirable in other embodiments to form the handpiece as a single combined unit, where the terms base unit and handle unit refer only to regions of an otherwise unified whole. All these variations, and others that would be apparent to one skilled in the art, are envisioned within the bounds of the claimed sterilizable vacuum handpiece.

### BRIEF DESCRIPTION OF THE DRAWINGS

Without limiting the scope of the sterilizable vacuum handpiece as claimed below and referring now to the drawings and figures, all shown not-to-scale:

FIG. 1 is an exploded cross-sectional view of an embodiment of the sterilizable vacuum handpiece;

FIG. 2 is an elevation view of the embodiment of FIG. 1;

FIG. 3 is a cross sectional view taken along the line 3-3 of FIG. 2;

FIG. 4 is a cross sectional view of an assembled base unit valve of an embodiment of the sterilizable vacuum handpiece taken along the line 4-4 of FIG. 2;

FIG. 5 is a cross sectional view of an assembled handle unit valve of an embodiment of the sterilizable vacuum handpiece taken along line 5-5 of FIG. 2;

FIG. 6 is an exploded cross sectional view of handle unit valve of an embodiment of the sterilizable vacuum handpiece;

FIG. 7 is a partially exploded elevated perspective view of an embodiment of the sterilizable vacuum handpiece;

FIG. 8 is elevated perspective view of an embodiment of a portion of the sterilizable vacuum handpiece;

FIG. 9 is an elevation view of an embodiment of a portion of the sterilizable vacuum handpiece;

FIG. 10 is an elevation view of an embodiment of a portion of the sterilizable vacuum handpiece;

FIG. 11 is an elevation view of an embodiment of a portion of the sterilizable vacuum handpiece;

FIG. 12 is an elevation view of an embodiment of a portion of the sterilizable vacuum handpiece;

FIG. 13 is an elevated perspective view of an embodiment of a portion of the sterilizable vacuum handpiece;

FIG. 14 is an elevated perspective view of an embodiment of a portion of the sterilizable vacuum handpiece;

FIG. 15 is an elevated perspective view of an embodiment of a portion of the sterilizable vacuum handpiece;

FIG. 16 is an elevation view of an embodiment of a portion of the sterilizable vacuum handpiece;

FIG. 17 is an elevation view of an embodiment of a portion of the sterilizable vacuum handpiece;

FIG. 18 is an elevation view of an embodiment of a portion of the sterilizable vacuum handpiece;

FIG. 19 is an elevation view of an embodiment of a portion of the sterilizable vacuum handpiece;

FIG. 20 is an elevation view of an embodiment of a portion of the sterilizable vacuum handpiece;

FIG. 21 is a cross sectional view of an embodiment of the sterilizable vacuum handpiece taken along line 21-21 of FIG. 16;

FIG. 22 is an elevation view of an embodiment of a portion of the sterilizable vacuum handpiece;

FIG. 23 is a cross sectional view of an embodiment of the sterilizable vacuum handpiece taken along line 23-23 of FIG. 17;

FIG. 24 is an exploded cross-sectional view of a base unit and base valve of an embodiment of the sterilizable vacuum handpiece;

FIG. 25 is a cross sectional view of an embodiment of the sterilizable vacuum handpiece taken along line 25-25 of FIG. 10;

FIG. 26 is a cross sectional view of an embodiment of the sterilizable vacuum handpiece taken along line 26-26 of FIG. 9;

FIG. 27 is an elevation view of an embodiment of the sterilizable vacuum handpiece; and

FIG. 28 is a cross sectional view of an embodiment of the sterilizable vacuum handpiece taken along line 28-28 of FIG. 27.

These drawings, all not-to-scale, are provided to assist in the understanding of the exemplary embodiments of the sterilizable vacuum handpiece as described in more detail below and should not be construed as unduly limiting the claimed sterilizable vacuum handpiece. In particular, the relative spacing, positioning, sizing and dimensions of the various elements illustrated in the drawings are not drawn to scale and may have been exaggerated, reduced or otherwise modified for the purpose of improved clarity. Those of ordinary skill in the art will also appreciate that a range of alternative configurations have been omitted simply to improve the clarity and reduce the number of drawings.

### DETAILED DESCRIPTION OF THE INVENTION

The claimed sterilizable vacuum handpiece (10) enables a significant advance in the state of the art. The preferred embodiments of the handpiece (10) accomplish this by new and novel arrangements of elements and methods that are configured in unique and novel ways and which demonstrate previously unavailable but preferred and desirable capabilities. The detailed description set forth below in connection with the drawings is intended merely as a description of the present embodiments of the claimed handpiece (10), and is not intended to represent the only form in which the handpiece (10) may be constructed or utilized. The description sets forth the designs, functions, means, and methods of implementing the handpiece (10) in connection with the illustrated embodiments. It is to be understood, however, that the same or equivalent functions and features may be accomplished by different embodiments that are also intended to be encompassed within the spirit and scope of the claimed handpiece (10).

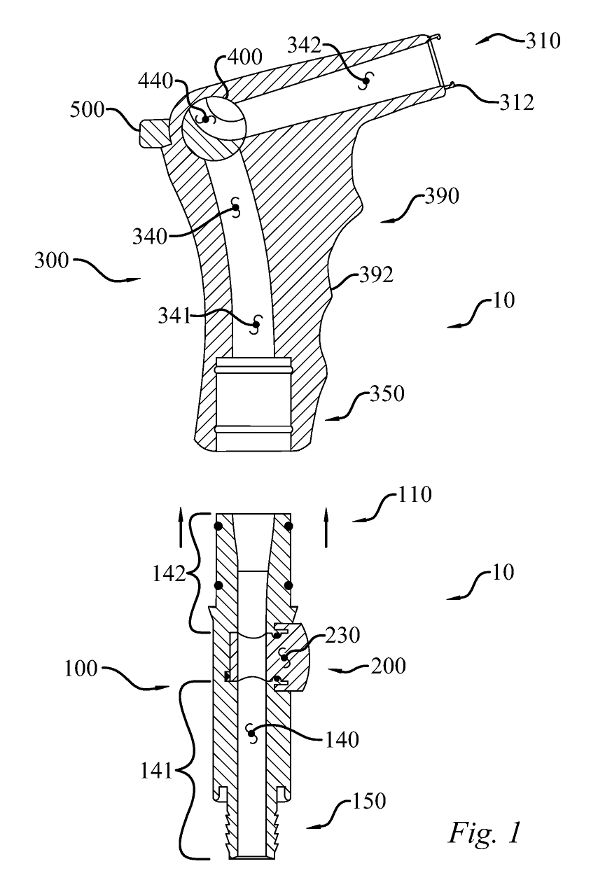

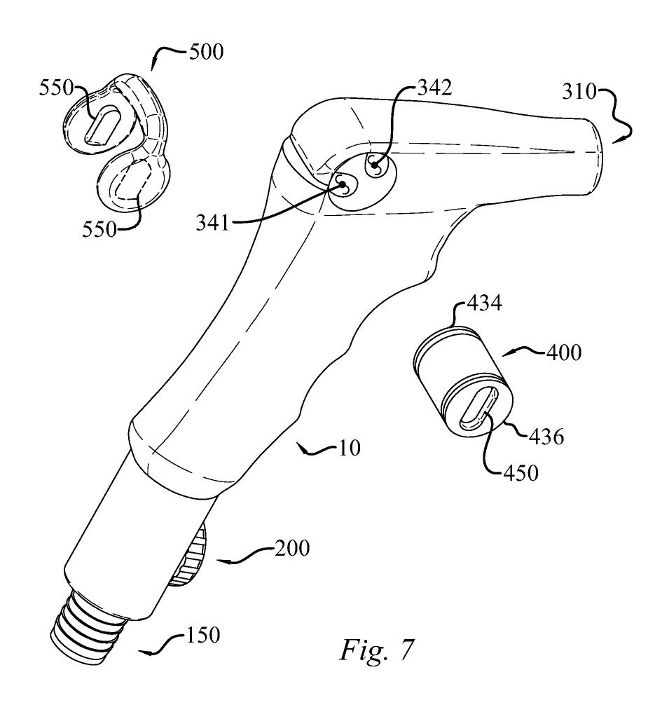

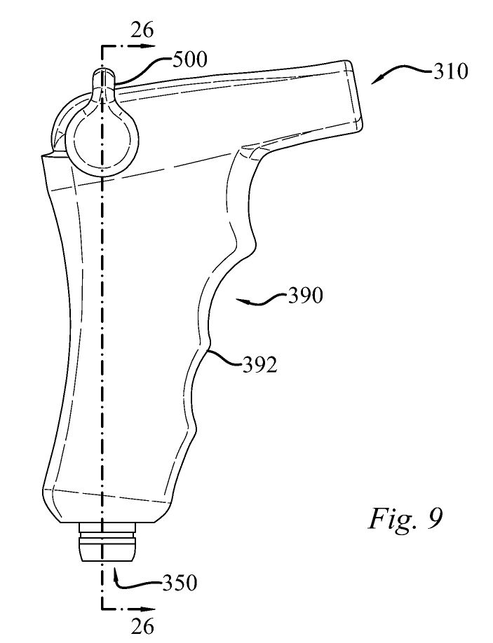

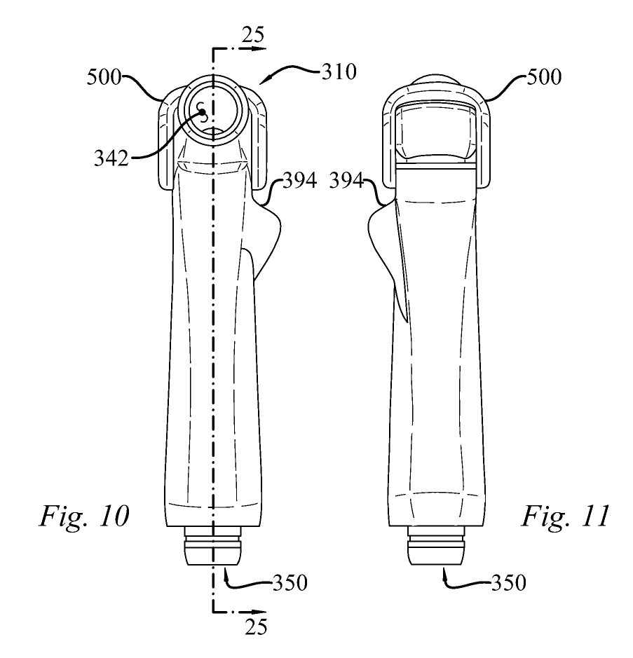

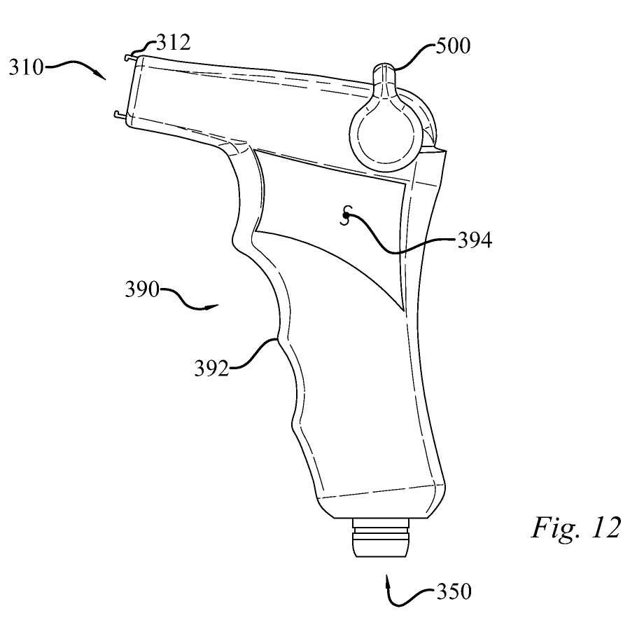

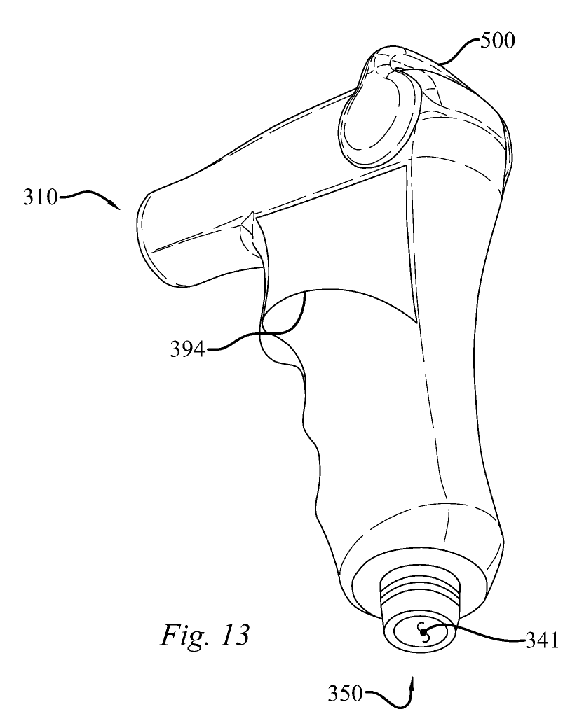

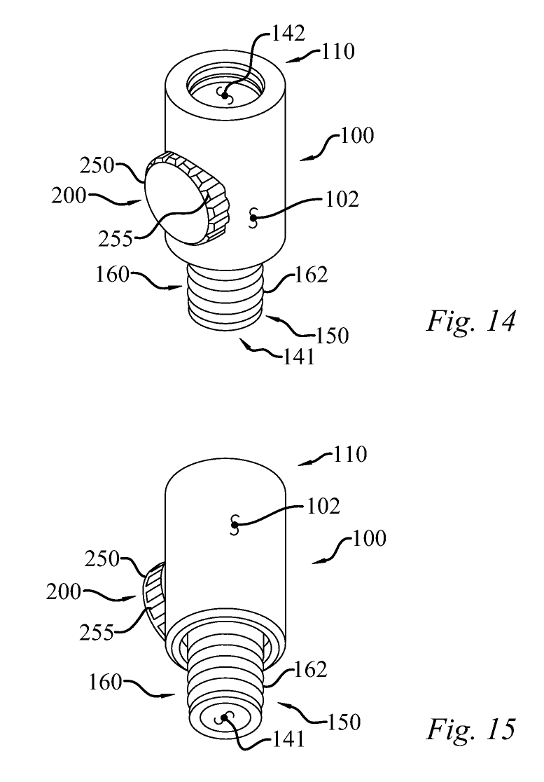

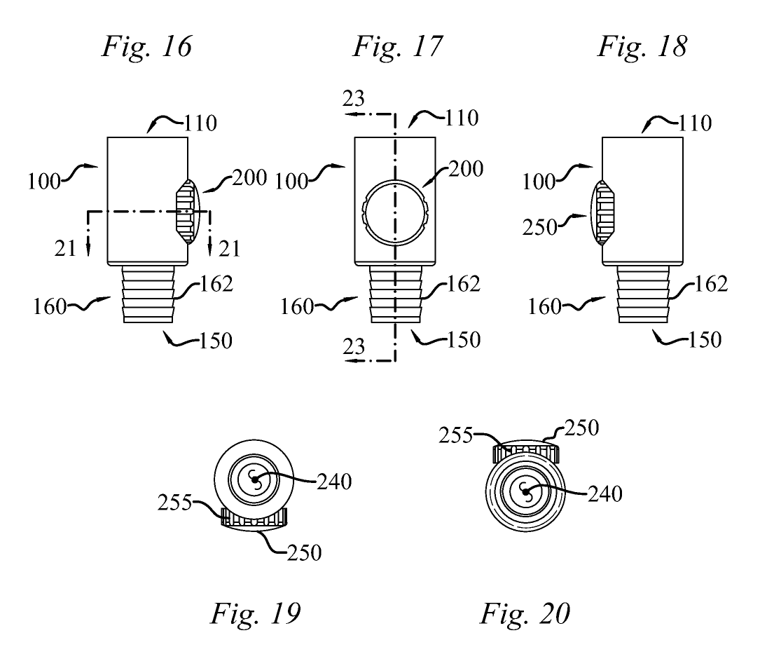

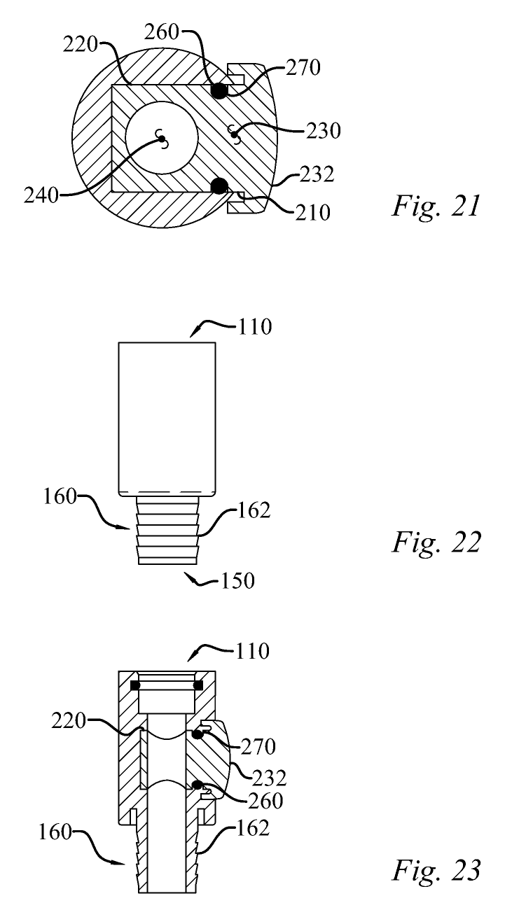

With regards to FIGS. 1-28, a sterilizable vacuum handpiece (10), in various embodiments may include a base unit (100) coupled to a handle unit (300), although by this it is not necessarily meant that the base unit (100) and the handle unit (300) be separable, and embodiments are specifically envisioned wherein the description of “units” merely identifies general regions of an integral, one-piece handpiece body. In one embodiment, generally seen in FIGS. 1-3, the base unit (100) may have a base unit external surface (102), base unit proximal end (110) and a base unit distal end (150), and an internally formed base unit vacuum passage (140) placing the base unit proximal end (110) and the base unit distal end (150) in adjustable fluid communication though the base unit (100).

Similarly, also seen generally in FIGS. 1-3, the handle unit (300) may have a handle unit external surface (302), a handle unit proximal end (310) and a handle unit distal end (350) and an internally formed handle unit vacuum passage (340) placing the handle unit proximal end (310) and the handle unit distal end (350) in fluid communication through the handle unit (300). No particular conformation is claimed as to the general shape and/or curvature, or lack of same, of the handle unit vacuum passage (340). As seen in FIG. 1, in at least one embodiment, the handle unit vacuum passage (340) has a slightly curvilinear shape, but may just as easily be straight, or have any other conformation as would be known to one skilled in the art.

In at least one embodiment, seen well in FIGS. 1, 2, 4, 21, 23, and 24, a hand-removable base unit valve (200) may separate the base unit vacuum passage (140) into a base unit vacuum passage proximal portion (142) in potential fluid communication with a base unit vacuum passage distal portion (141). By hand-removable, it is meant that the normal finger strength of a human operator is sufficient to remove and replace the base unit valve (200) in its position in the base unit (100). The base unit valve (200) may control the adjustable fluid communication between the base unit vacuum passage proximal portion (142) and the base unit vacuum passage distal portion (141).

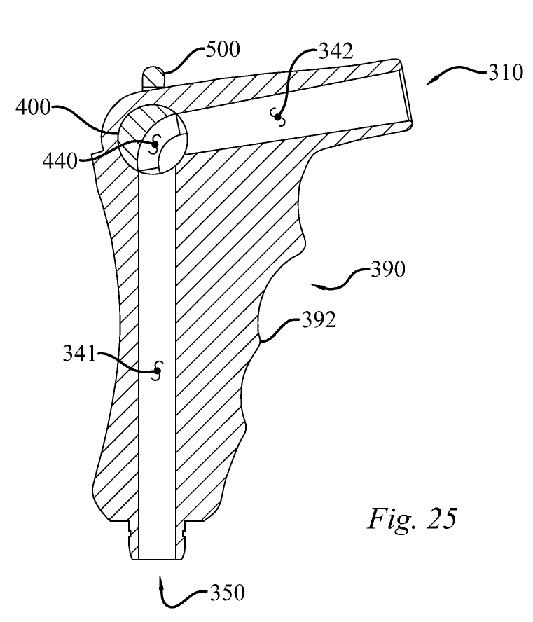

Again, in at least one embodiment, a hand-removable handle unit valve (400), seen well in FIGS. 1, 2, and 5, may separate the handle unit vacuum passage (340) into a handle unit vacuum passage proximal portion (342) in potential fluid communication with a handle unit vacuum passage distal portion (341). No particular conformation is claimed as to the general shape and/or curvature, or lack of same, of the handle unit vacuum passage distal portion (341). As seen in FIG. 25, in at least one embodiment, the handle unit vacuum passage distal portion (341) is generally straight, but may just as easily be slightly curvilinear in shape, or have any other conformation as would be known to one skilled in the art.

As was the case with the base unit valve (200), by hand-removable, it is meant that the normal finger strength of a human operator is sufficient to remove and replace the handle unit valve (400) in its position in the handle unit (300). The handle unit valve (400) may control the fluid communication between the handle unit vacuum passage proximal portion (342) and the handle unit vacuum passage distal portion (341).

One skilled in the art will appreciate that functional combination of the base unit valve (200) and the handle unit valve (400) may confer certain advantages in various embodiments. For example, and without intending any limitation, the base unit valve (200) may control the overall maximum suction flow through the handpiece (10) while the handle unit valve (400) placed in an ergonomically convenient location, can be used to make subtle and moment-by-moment variations in the amount of suction actually deployed.

In a further embodiment, the handle unit valve stem (430), seen in FIGS. 1, 5, 6, and 26, may be substantially cylindrical and the handle unit vacuum passage (340) is a curvilinear passage having at least a first sidewall length lesser in length than at least a second sidewall length.

One skilled in the art will realize that numerous valve configurations are possible. In one embodiment, the base unit valve (200), seen well in FIGS. 1, 2, 4 and 24, further includes a base unit valve bore (210) having at least one base unit valve bore surface (212) extending from the base unit external surface (102) to the base unit vacuum passage (140). This construction may thereby place the base unit external surface (102) in communication with the base unit vacuum passage (140), with the base unit valve bore (210) reversibly receiving a base unit valve stem (230) having at least one base unit valve stem surface (232). In such an embodiment, the base unit valve stem (230) may be rotatably received within the base unit valve bore (210), and the base unit valve stem (230) may have an internally formed base unit valve vacuum passage (240). Rotation of the base unit valve stem (230) may place the base unit vacuum passage proximal portion (142) and the base unit vacuum passage distal portion (141) in adjustable fluid communication.

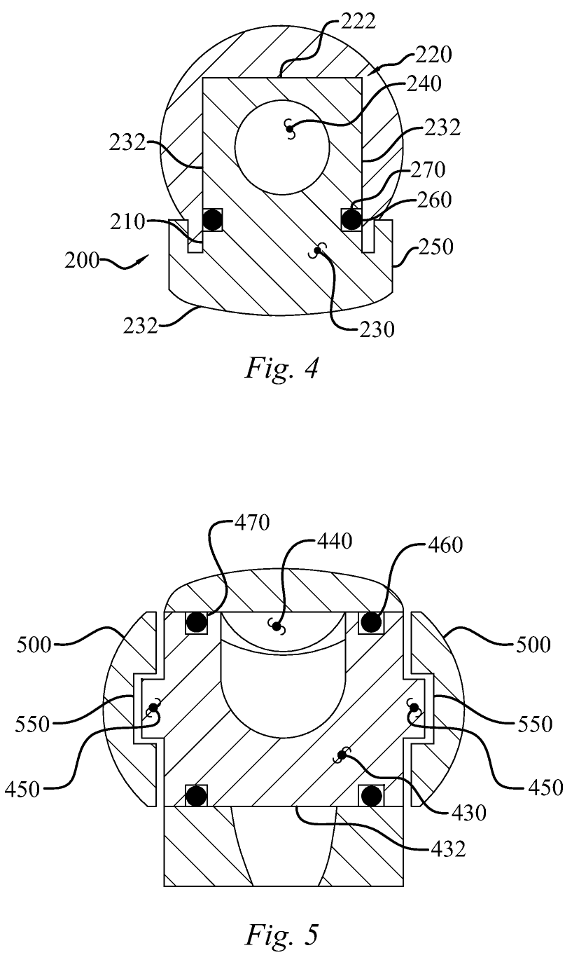

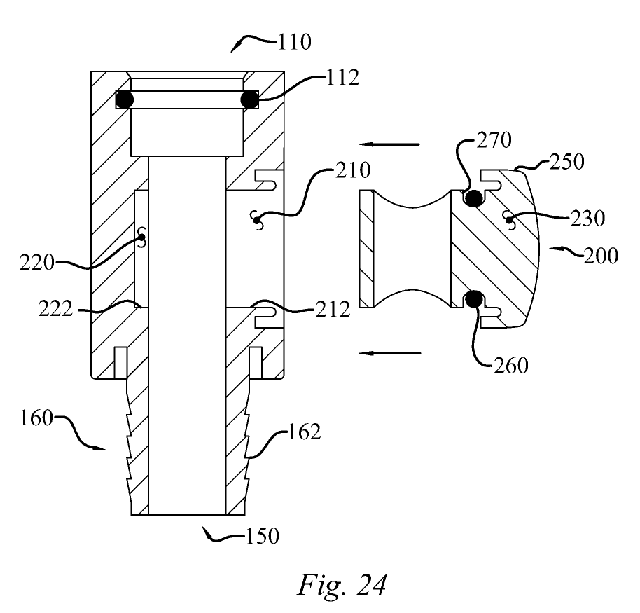

In yet another embodiment, also seen well in FIGS. 4 and 24, the base unit valve (200) may further include a base unit valve counter-bore (220) having at least one base unit valve counter-bore surface (222), diametrically opposite to the base unit valve bore (210). Such a base unit valve counter-bore (220) may extend a partial distance between the base unit vacuum passage (140) and the base unit external surface (102), with the base unit valve counter-bore (220) being sized to reversibly receive a portion of the base unit valve stem (230). In such an embodiment, a portion of the base unit valve stem (230) may be rotatably received within the base unit valve counter-bore (220). The base unit valve stem (230) may be releasably retained by friction fit within the base unit valve bore (210) and the base unit valve counter-bore (220), in keeping with a general constructional aspiration that all valves (200, 400) be hand-removable.

In order to maximize a fluid-tight fit between all parts of the handpiece (10), in some embodiments, the base unit valve stem surface (232) may have at least one base unit valve O-ring retainer (270) sized to contain at least one base unit valve O-ring (260), seen well in FIG. 24. It is equally possible for the base unit valve bore surface (212) to have at least one base unit valve O-ring retainer (270) sized to contain at least one base unit valve O-ring (260).

One skilled in the art will realize that there will be numerous surface area relationships possible between a base unit valve vacuum passage (240) surface area and a base unit vacuum passage (140) surface area, but in at least one embodiment, the base unit valve vacuum passage (240) may have a surface area equal to or greater than 80% of a smallest base unit vacuum passage (140) surface area, it being recognized that the base unit vacuum passage (140) may not have a uniform diameter nor a uniform surface area.

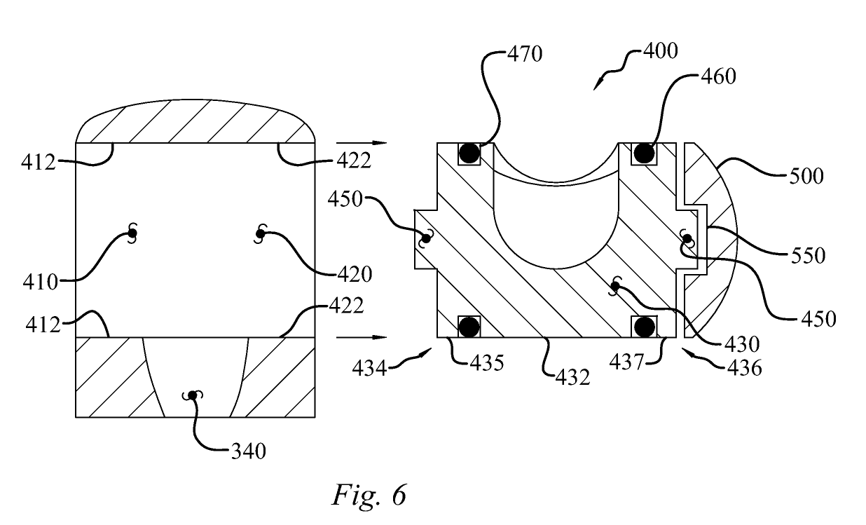

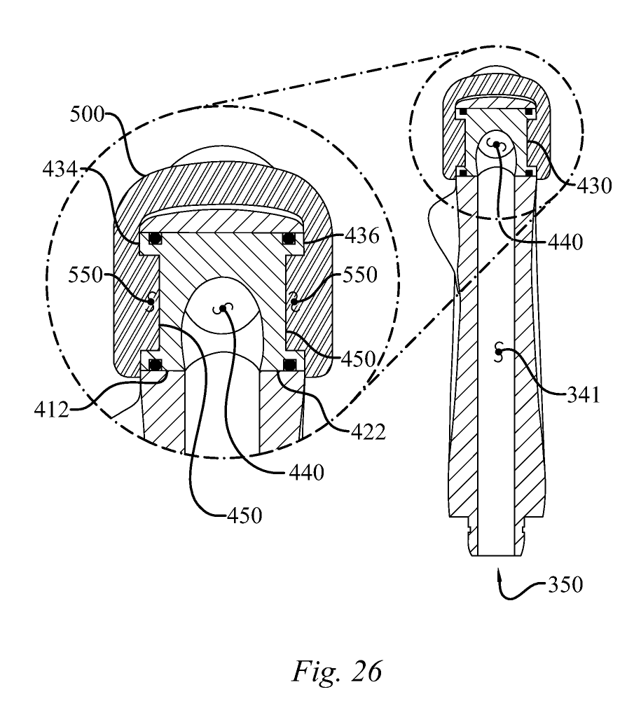

One skilled in the art will also realize that numerous possible designs are feasible for the handle unit valve (400). In one embodiment, seen well in FIGS. 6 and 26, the handle unit valve (400) may further include a first handle unit valve bore (410) having at least one first handle unit valve bore surface (412) extending from the handle unit external surface (302) to the handle unit vacuum passage (340). In such an embodiment, the first handle unit valve bore (410) may reversibly receive a handle unit valve stem (430) having a handle unit valve stem surface (432). There may be a second handle unit valve bore (420) having at least one second handle unit valve bore surface (422) on an opposing surface of the handle unit (300) from the first handle unit valve bore (410), extending from the handle unit external surface (302) to the handle unit vacuum passage (340). Such a second handle unit valve bore (420) may also reversibly receive the handle unit valve stem (430), and the handle unit valve stem (430) may be rotatably received within the first handle unit valve bore (410) and the second handle unit valve bore (420).

The handle unit valve stem (430), also seen well in FIGS. 1 and 26, may have an internally formed handle unit valve vacuum passage (440), whereby rotation of the handle unit valve stem (430) places the handle unit vacuum passage proximal portion (342) and the handle unit vacuum passage distal portion (341) in adjustable fluid communication. Thus the operator has, in a convenient and ergonomic position, means for making very subtle adjustments on a near continuous basis in flow through the handpiece (10).

As with the base unit valve (200), one skilled in the art will realize that there will be numerous surface area relationships possible between a handle unit valve vacuum passage (440) surface area and a handle unit vacuum passage (340) surface area, but in one embodiment, intended at least in part to minimize the chances of obstruction at the handle unit valve (400), the handle unit valve vacuum passage (440) may have a surface area equal to or greater than 80% of a smallest handle unit vacuum passage (340) surface area, it being recognized that the handle unit vacuum passage (340) may not have a uniform diameter nor a uniform surface area.

In order to maximize a fluid-tight fit between all parts of the handpiece (10), in some embodiments, the handle unit valve stem surface (432) may have at least one handle unit valve O-ring retainer (470) sized to contain at least handle unit valve O-ring (460), seen well in FIG. 6. Equally possible, the first handle unit valve bore surface (412) and/or the second handle unit valve bore surface (422) may have at least one handle unit valve O-ring retainer (470) sized to contain at least one handle unit valve O-ring (460), seen well in FIG. 26.

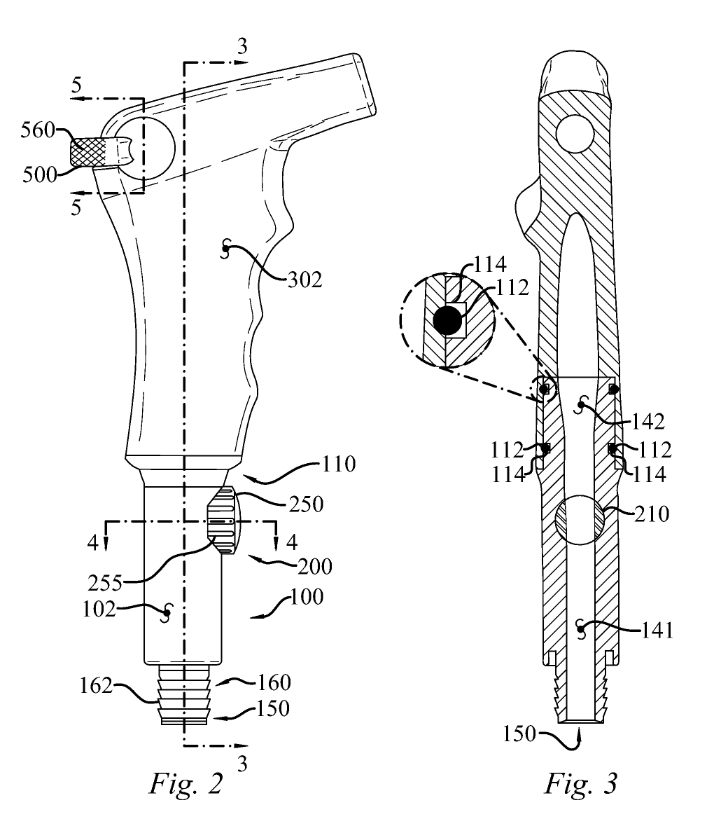

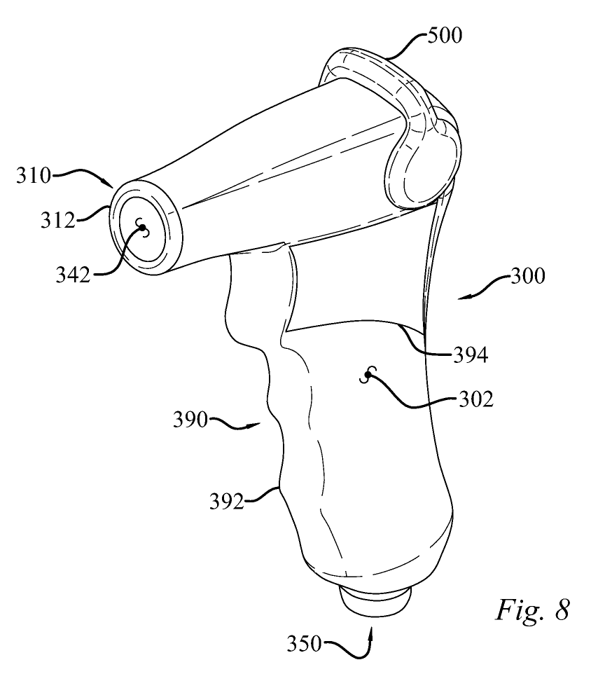

Numerous other variations and embodiments will be found useful by one skilled in the art. For example, the base unit distal end (150) may further have a releasable vacuum connection (160) for attaching the instant invention to a wide variety of suction sources, seen well in FIG. 2. To improve hand feel and dexterity while using the handpiece, the base unit valve (200) may further comprise a base unit valve finger grip (250), as seen in FIG. 2, and the handle unit external surface (302) may have at least one ergonomic gripping means selected from the group of gripping means consisting of finger ridges (392) and at least one thumb projection (394), seen well in FIGS. 8-12.

While numerous designs are feasible for the handle unit valve (400), in one preferred embodiment, seen well in FIGS. 6 and 26, the handle unit valve stem (430) may have a handle unit valve stem first end (434) having a handle unit valve stem controller retainer (450) and a handle unit valve stem second end (436) having a handle unit valve stem controller retainer (450). In such an embodiment, there may be a controller (500) having at least one controller-handle unit valve stem retainer (550), reversibly joined to the handle unit valve stem first end (434) and the handle unit valve stem second end (436), by means of cooperation between the at least one controller-handle unit valve stem retainer (550) and at least one of the handle unit valve stem controller retainers (450).

In at least one embodiment, patterned on that seen in FIGS. 6 and 26, the handle valve (400) may thereby be releasably retained by the opposing ends of a “bucket-handle” shaped controller (500), which may be easily removed to allow for removal of the handle unit valve stem (430) for cleaning. In general, ease of cleaning and sterilization is one of the major features of many of the preferred embodiments of the handpiece (10). The valves (200, 400) are hand removable, and may be easily slipped from the handpiece (10). The handpiece (10) may be further disassembled into the base unit (100) and the handle unit (300) for cleaning and sterilization. In one embodiment, the base unit (100) may have at least one base unit proximal end O-ring retainer (114) sized to contain at least one base unit proximal end O-ring (112), as seen in FIG. 3. However, as a more general teaching, O-rings (112, 260, 460) held in corresponding O-ring retainers (114, 270, 470) encourage fluid-tight seals at the valves (200, 400) while still allowing easy disassembly for cleaning and sterilization.

Furthermore, while it may be illustrated in this specification that O-rings (112, 260, 460) and corresponding retainers (114, 270, 470) appear on a particular part of the handpiece (10), there is no reason why these structures cannot appear on the corresponding parts of the handpiece (10). For example, while the base unit proximal end (110) may be depicted, as seen well in FIGS. 1 and 2, as a male fitting bearing at least one base unit proximal end O-ring (112) retained within at least one base unit proximal end O-ring retainer (114), there is no reason why the handpiece cannot be configured such that the at least one O-ring (112) and O-ring retainer (114) could not be placed on an internal surface of the handle unit vacuum passage (340). Furthermore, while the base unit (100) may be depicted, as seen well also in FIGS. 1 and 2, as a male fitting at the base unit proximal end (110) and the handle unit (300) as a female fitting at the handle unit distal end (350), there is no reason why this relationship could not be inverted, providing a female fitting on the base unit (100) at the base unit proximal end (110) and a male fitting on the handle unit (300) at the handle unit distal end (350).

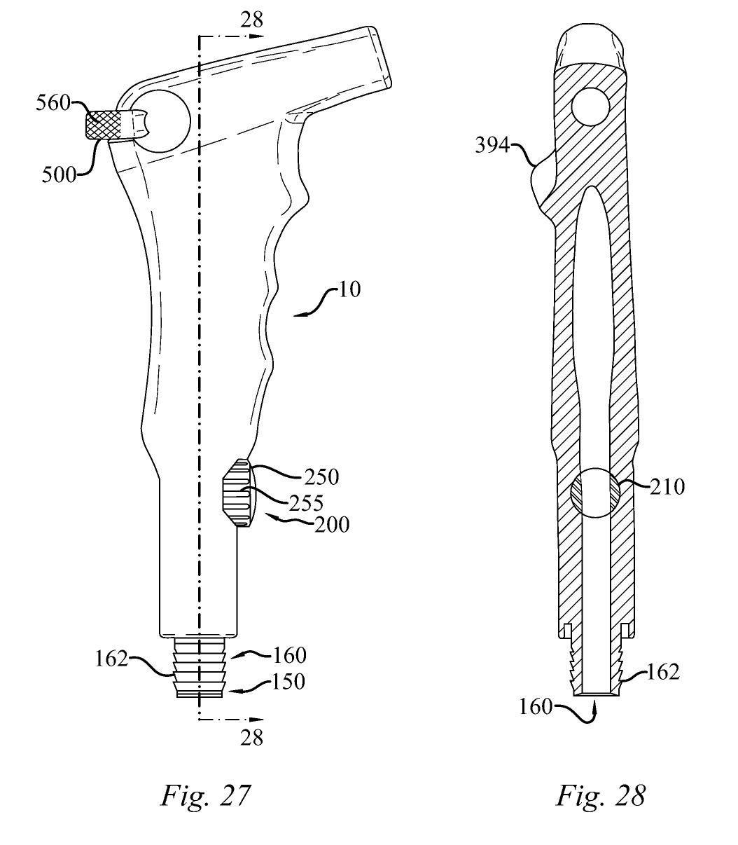

Additionally, while in a preferred embodiment, the base unit (100) and the handle unit (300) may be reversibly coupled in a liquid-tight fit, it may be desirable in other embodiments, seen by way of example only in FIG. 27-28, to form the handpiece (10) as a single combined unit, where the terms base unit (100) and handle unit (300) refer only to regions of an otherwise unified structure. All these variations, and others that would be apparent to one skilled in the art, are envisioned within the bounds of the claimed handpiece (10).

Other embodiments will be found advantageous by one skilled in the art, such as providing releasable tip attachment means (312) at the handle unit proximal end (310) to allow for the releasable attachment of a wide variety of specialized disposable and non-disposable tips, according to the particular needs of certain uses of the handpiece (10).

Numerous alterations, modifications, and variations of the preferred embodiments disclosed herein will be apparent to those skilled in the art and they are all anticipated and contemplated to be within the spirit and scope of the claimed handpiece (10). Further, although specific embodiments have been described in detail, those with skill in the art will understand that the preceding embodiments and variations can be modified to incorporate various types of substitute and or additional or alternative materials, relative arrangement of elements, and dimensional configurations. Accordingly, even though only few variations of the handpiece (10) are described herein, it is to be understood that the practice of such additional modifications and variations and the equivalents thereof, are within the spirit and scope of the handpiece (10) as defined in the following claims.

I claim:

1. A sterilizable vacuum handpiece (10) comprising;
a base unit (100) coupled to a handle unit (300), wherein,
a) the base unit (100) has a base unit external surface (102), base unit proximal end (110) and a base unit distal end (150), and an internally formed base unit vacuum passage (140) placing the base unit proximal end (110) and the base unit distal end (150) in adjustable fluid communication through the base unit (100), and
b) the handle unit (300) has a handle unit external surface (302), a handle unit proximal end (310) and a handle unit distal end (350) and an internally formed handle unit vacuum passage (340) placing the handle unit proximal end (310) and the handle unit distal end (350) in fluid communication through the handle unit (300),
a hand-removable base unit valve (200) separating the base unit vacuum passage (140) into a base unit vacuum passage proximal portion (142) in adjustable fluid communication with a base unit vacuum passage distal portion (141) and wherein the base unit valve (200) controls the adjustable fluid communication between the base unit vacuum passage proximal portion (142) and the base unit vacuum passage distal portion (141),
a hand-removable handle unit valve (400) separating the handle unit vacuum passage (340) into a handle unit vacuum passage proximal portion (342) in potential fluid communication with a handle unit vacuum passage distal portion (341) and wherein the handle unit valve (400) controls the adjustable fluid communication between the handle unit vacuum passage proximal portion (342) and the handle unit vacuum passage distal portion (341).

2. The sterilizable vacuum handpiece (10) of claim 1, wherein the handle unit valve (400) has a handle unit valve stem (430) that is substantially cylindrical and the handle unit vacuum passage (340) is a curvilinear passage having at least a first sidewall length lesser in length than at least a second sidewall length.

3. The sterilizable vacuum handpiece (10) of claim 1, wherein the base unit valve (200) further comprises,
a base unit valve bore (210) having at least one base unit valve bore surface (212) extending from the base unit external surface (102) to the base unit vacuum passage (140), thereby placing the base unit external surface (102) in communication with the base unit vacuum passage (140), the base unit valve bore (210) reversibly receiving a base unit valve stem (230) having at least one base unit valve stem surface (232),
wherein the base unit valve stem (230) is rotatably received within the base unit valve bore (210), and
the base unit valve stem (230) has an internally formed base unit valve vacuum passage (240), and wherein rotation of the base unit valve stem (230) places the base unit vacuum passage proximal portion (142) and the base unit vacuum passage distal portion (141) in adjustable fluid communication.

4. The sterilizable vacuum handpiece (10) of claim 3, wherein the base unit valve (200) further comprises,
a base unit valve counter-bore (220) having at least one base unit valve counter-bore surface (222), diametrically opposite to the base unit valve bore (210) and extending a partial distance between the base unit vacuum passage (140) and the base unit external surface (102), the base unit valve counter-bore (220) being sized to reversibly receive a portion of the base unit valve stem (230),
wherein a portion of the base unit valve stem (230) is rotatably received within the base unit valve counter-bore (220), and
wherein the base unit valve stem (230) is releasably retained by friction fit within the base unit valve bore (210) and the base unit valve counter-bore (220).

5. The sterilizable vacuum handpiece (10) of claim 3, wherein the at least one base unit valve stem surface (232) has at least one base unit valve O-ring retainer (270) sized to contain at least one base unit valve O-ring (260).

6. The sterilizable vacuum handpiece (10) of claim 3, wherein the at least one base unit valve bore surface (212) has at least one base unit valve O-ring retainer (270) sized to contain at least one base unit valve O-ring (260).

7. The sterilizable vacuum handpiece (10) of claim 3, wherein the base unit valve vacuum passage (240) has a surface area equal to or greater than 80% of a smallest base unit vacuum passage (140) surface area.

8. The sterilizable vacuum handpiece (10) of claim 1, wherein the handle unit valve (400) further comprises,
a first handle unit valve bore (410) having at least one first handle unit valve bore surface (412) extending from the handle unit external surface (302) to the handle unit vacuum passage (340), the first handle unit valve bore (410) reversibly receiving a handle unit valve stem (430) having a handle unit valve stem surface (432), and
a second handle unit valve bore (420) having at least one second handle unit valve bore surface (422) on an opposing surface of the handle unit (300) from the first handle unit valve bore (410), extending from the handle unit external surface (302) to the handle unit vacuum passage (340), the second handle unit valve bore (420) reversibly receiving the handle unit valve stem (430), and
wherein the handle unit valve stem (430) is rotatably received within the first handle unit valve bore (410) and the second handle unit valve bore (420), and
the handle unit valve stem (430) has an internally formed handle unit valve vacuum passage (440), and wherein rotation of the handle unit valve stem (430) places the handle unit vacuum passage proximal portion (342) and the handle unit vacuum passage distal portion (341) in adjustable fluid communication.

9. The sterilizable vacuum handpiece (10) of claim 8, wherein the handle unit valve vacuum passage (440) has a surface area equal to or greater than 80% of a smallest handle unit vacuum passage (340) surface area.

10. The sterilizable vacuum handpiece (10) of claim 8, wherein the handle unit valve stem surface (432) has at least one handle unit valve O-ring retainer (470) sized to contain at least one handle unit valve O-ring (460).

11. The sterilizable vacuum handpiece (10) of claim 8, wherein at least one of the handle unit valve bore surfaces (412, 422) selected from the group of surfaces consisting of the first handle unit valve bore surface (412) and the second handle unit valve bore surface (422), has at least one handle unit valve O-ring retainer (470) sized to contain at least one handle unit valve O-ring (460).

12. The sterilizable vacuum handpiece (10) of claim 1, wherein the base unit valve (200) further comprises a base unit valve finger grip (250).

13. The sterilizable vacuum handpiece (10) of claim 1, wherein the handle unit external surface (302) further comprises at least one ergonomic gripping means selected from the group of gripping means consisting of finger ridges (392) and at least one thumb projection (394).

14. The sterilizable vacuum handpiece (10) of claim 8, wherein the handle unit valve stem (430) has a handle unit valve stem first end (434) having a handle unit valve stem controller retainer (450) and a handle unit valve stem second end (436) having a handle unit valve stem controller retainer (450).

15. The sterilizable vacuum handpiece (10) of claim 14, further comprising a controller (500) having at least one controller-handle unit valve stem retainer (550), reversibly joined to the handle unit valve stem first end (434) and the handle unit valve stem second end (436) by means of cooperation between the at least one controller-handle unit valve stem retainer (550) and at least one of the handle unit valve stem controller retainers (450).

16. The sterilizable vacuum handpiece (10) of claim 1, wherein the base unit (100) has at least one base unit proximal end O-ring retainer (114) sized to contain at least one base unit proximal end O-ring (112).

17. The sterilizable vacuum handpiece (10) of claim 1, wherein the handle unit proximal end (310) further comprises releasable tip attachment means (312).

18. The sterilizable vacuum handpiece (10) of claim 1, wherein the base unit (100) and the handle unit (300) are reversibly coupled in a liquid-tight fit.

19. A sterilizable vacuum handpiece (10) comprising;
a base unit (100) coupled to a handle unit (300), wherein the base unit (100) has a base unit external surface (102), base unit proximal end (110) and a base unit distal end (150), and an internally formed base unit vacuum passage (140) placing the base unit proximal end (110) and the base unit distal end (150) in adjustable fluid communication through the base unit (100), and
the handle unit (300) has a handle unit external surface (302), a handle unit proximal end (310) and a handle unit distal end (350) and an internally formed handle unit vacuum passage (340) placing the handle unit proximal end (310) and the handle unit distal end (350) in fluid communication through the handle unit (300),
a hand-removable base unit valve (200) separating the base unit vacuum passage (140) into a base unit vacuum passage proximal portion (142) in potential fluid communication with a base unit vacuum passage distal portion (141) and wherein the base unit valve (200) controls the adjustable fluid communication between the base unit vacuum passage proximal portion (142) and the base unit vacuum passage distal portion (141), wherein the base unit valve (200) further comprises,
a base unit valve bore (210) having at least one base unit valve bore surface (212) extending from the base unit external surface (102) to the base unit vacuum passage (140) thereby placing the base unit external surface (102) in communication with the base unit vacuum passage (140), the base unit valve bore (210) reversibly receiving a base unit valve stem (230) having at least one base unit valve stem surface (232),
wherein the base unit valve stem (230) is rotatably received within the base unit valve bore (210),
the base unit valve stem (230) having an internally formed base unit valve vacuum passage (240), and wherein rotation of the base unit valve stem (230) places the base unit vacuum passage proximal portion (142) and the base unit vacuum passage distal portion (141) in adjustable fluid communication,
a base unit valve counter-bore (220) having at least one base unit valve counter-bore surface (222), diametrically opposite to the base unit valve bore (210) and extending a partial distance between the base unit vacuum passage (140) and the base unit external surface (102), the base unit valve counter-bore (220) being sized to reversibly receive a portion of the base unit valve stem (230),
wherein a portion of the base unit valve stem (230) is rotatably received within the base unit valve counter-bore (220), and
wherein the base unit valve stem (230) is releasably retained by friction fit within the base unit valve bore (210) and the base unit valve counter-bore (220), and
a hand-removable handle unit valve (400) separating the handle unit vacuum passage (340) into a handle unit vacuum passage proximal portion (342) in potential fluid communication with a handle unit vacuum passage distal portion (341) and wherein the handle unit valve (400) controls the fluid communication between the handle unit vacuum passage proximal portion (342) and the handle unit vacuum passage distal portion (341).

20. A sterilizable vacuum handpiece (10) comprising;
a base unit (100) coupled to a handle unit (300), wherein the base unit (100) has a base unit external surface (102), base unit proximal end (110) and a base unit distal end (150), and an internally formed base unit vacuum passage (140) placing the base unit proximal end (110) and the base unit distal end (150) in adjustable fluid communication through the base unit (100), and
the handle unit (300) has a handle unit external surface (302), a handle unit proximal end (310) and a handle unit distal end (350) and an internally formed handle unit vacuum passage (340) placing the handle unit proximal end (310) and the handle unit distal end (350) in fluid communication through the handle unit (300),
a hand-removable base unit valve (200) separating the base unit vacuum passage (140) into a base unit vacuum passage proximal portion (142) in potential fluid communication with a base unit vacuum passage distal portion (141) and wherein the base unit valve (200) controls the adjustable fluid communication between the base unit vacuum passage proximal portion (142) and the base unit vacuum passage distal portion (141),
a hand-removable handle unit valve (400) separating the handle unit vacuum passage (340) into a handle unit vacuum passage proximal portion (342) in potential fluid communication with a handle unit vacuum passage distal portion (341) and wherein the handle unit valve (400) controls the fluid communication between the handle unit vacuum passage proximal portion (342) and the handle unit vacuum passage distal portion (341), wherein the handle unit valve (400) further comprises,
a first handle unit valve bore (410) having at least one first handle unit valve bore surface (412) extending from the handle unit external surface (302) to the handle unit vacuum passage (340), the first handle unit valve bore (410) reversibly receiving a handle unit valve stem (430) having a handle unit valve stem surface (432), and
a second handle unit valve bore (420) having at least one second handle unit valve bore surface (422) on an opposing surface of the handle unit (300) from the first handle unit valve bore (410), extending from the handle unit external surface (302) to the handle unit vacuum passage (340), the second handle unit valve bore (420) reversibly receiving the handle unit valve stem (430), and
wherein the handle unit valve stem (430) is rotatably received within the first handle unit valve bore (410) and the second handle unit valve bore (420), and
the handle unit valve stem (430) has an internally formed handle unit valve vacuum passage (440), and wherein rotation of the handle unit valve stem (430) places the handle unit vacuum passage proximal portion (342) and the handle unit vacuum passage distal portion (341) in adjustable fluid communication.
